(19) United States
(12) Patent Application Publication
(10) Pub. No.: US 2026/0091036 A1
(43) Pub. Date: Apr. 2, 2026

(54) Dosing Regimens for Elagolix

(71) Applicant: AbbVie Inc., North Chicago, IL (US)

(72) Inventor: Mohamad Shebley, Gurnee, IL (US)

(21) Appl. No.: 19/310,386

(22) Filed: Aug. 26, 2025

Related U.S. Application Data
(63) Continuation of application No. 18/494,554, filed on Oct. 25, 2023, now abandoned.
Application No. 18/494,554 is a continuation of application No. 17/263,009, now abandoned.
(60) Provisional application No. 62/713,243, filed on Aug. 1, 2018.
(60) Provisional application No. 62/719,459, filed on Aug. 17, 2018.

(51) Int. Cl. A61K 31/513; A61K 31/565; A61K 31/567; A61P 15/00; A61P 19/00

(52) U.S. Cl. CPC A61K 31/513; A61K 31/565; A61K 31/567; A61P 15/00; A61P 19/00

(57) ABSTRACT

The present invention relates to dosing regimens for GnRH receptor antagonists, and, in particular, dosing regimens for 4-((R)-2-[5-(2-fluoro-3-methoxy-phenyl)-3-(2-fluoro-6trifluoromethyl-benzyl)-4-methyl-2,6-dioxo-3,6-dihydro-2H-pyrimidin-1-yl]-1-phenylethylamino)-butyric acid (Compound A) or a pharmaceutically acceptable salt thereof, in subjects suffering from, for example, endometriosis, adenomyosis, polycystic ovary syndrome (PCOS), or uterine fibroids, to minimize changes in bone mineral density associated with such GnRH receptor antagonists.

### TECHNICAL FIELD

The present invention relates to dosing regimens for GnRH receptor antagonists in subjects suffering from, for example, endometriosis, adenomyosis, polycystic ovary syndrome (PCOS), or uterine fibroids and, in particular, to dosing regimens to minimize changes in bone mineral density associated with such GnRH receptor antagonists.

### BACKGROUND

Endometriosis is a disease in which tissue normally found in the uterine cavity (i.e., endometrium) is found outside the uterus, usually implanted on the peritoneal lining of the pelvis. Endometriosis affects an estimated 1 in 10 women of reproductive age and can cause pain, infertility, and sexual dysfunction. Growth of endometrial tissue outside of the uterine cavity is believed to be estrogen-dependent. Thus, therapies for endometriosis have been aimed at altering estrogen levels.

Adenomyosis is an estrogen-dependent disease of benign endometrial tissue growth within the uterine muscular tissue, and is associated with heavy menstrual bleeding (FMB; menorrhagia, defined as greater than 80 mL per menstrual cycle) (The Menorrhagia Research Group. Quantification of menstrual blood loss. The Obstetrician & Gynaecologist. 2004; 6:88-92) and dysmenorrhea. Adenomyosis occurs when endometrial tissue, which normally lines the uterus, exists within and grows into the muscular wall of the uterus. The displaced endometrial tissue continues to act as it normally would—thickening, breaking down and bleeding—during each menstrual cycle. An enlarged uterus and painful, heavy periods can result. Symptoms most often start late in the childbearing years after having children. The cause of adenomyosis remains unknown, but the disease typically disappears after menopause. For women who experience severe discomfort from adenomyosis, certain treatments can help, but hysterectomy is the only cure. Sometimes, adenomyosis is silent—causing no signs or symptoms—or only mildly uncomfortable. In other cases, adenomyosis may cause: heavy or prolonged menstrual bleeding, severe cramping or sharp, knifelike pelvic pain during menstruation (dysmenorrhea), menstrual cramps that last throughout menstruation, pain during intercourse, and blood clots that pass during menstruation.

Polycystic ovary syndrome (PCOS) is a hormonal disorder common among women of reproductive age. Women with PCOS may have infrequent or prolonged menstrual periods or excess male hormone (androgen) levels. The ovaries may develop numerous small collections of fluid (follicles) and fail to regularly release eggs.

Uterine fibroids (leiomyomas) are benign tumors and are highly prevalent in women of reproductive age. Symptoms associated with uterine fibroids most commonly include heavy or prolonged menstrual bleeding, pelvic pressure and pelvic organ compression, back pain, and adverse reproductive outcomes. Heavy menstrual bleeding is inconvenient and may lead to iron-deficiency anemia, which is the leading cause of surgical interventions that may include hysterectomy. Other symptoms, in particular pressure symptoms, are largely dependent on the size, number, and location of the tumors.

Although the pathogenesis has yet to be fully elucidated, the growth of uterine fibroids is known to be highly dependent on both estrogen and progestogen. Fibroids tend to shrink after menopause due to a decrease in hormone production. On this basis, most medical treatments for women with symptomatic uterine fibroids are aimed at either hormone-blocking or hormone-modulating strategies.

Gonadotropin-releasing hormone (GnRH) is a peptide that stimulates the secretion of the pituitary hormones that are responsible for sex steroid production and normal reproductive function. GnRH agonists are used to treat endometriosis and uterine fibroids by suppressing the activity of the pituitary-gonadal axis. However, GnRH agonists cause an initial stimulation of gonadotropic and gonadal hormones, such as estrogen.

Peptide GnRH antagonists competitively bind to GnRH receptors in the pituitary gland, blocking the release of gonadotropins, such as luteinizing hormone (LH) and follicle-stimulating hormone (FSH) from the pituitary. Such peptide GnRH antagonists have been approved for oncology and assisted reproduction. However, administration is inconvenient, with peptide GnRH antagonists being delivered as daily subcutaneous injections or as long-acting depot formulations.

An orally-administered, nonpeptide small molecule GnRH receptor antagonist, elagolix, has recently been approved for the management of moderate to severe pain associated with endometriosis. Elagolix may cause dose-dependent decreases in bone mineral density (BMD). Moreover, BMD loss is greater with increasing duration of use and may not be completely reversible after stopping treatment. The current prescribing information for elagolix indicates that the duration of use should be limited because of bone loss. The approved dosage and administration indicates that patients with normal liver function or mild hepatic impairment should receive 150 mg once daily for up to 24 months or 200 mg twice daily for up to 6 months.

Thus, there is a need in the art for new treatments and treatment regimens for endometriosis, adenomyosis, polycystic ovary syndrome (PCOS), uterine fibroids and, in particular, management of pain associated and/or heavy menstrual bleeding associated with such diseases. Moreover, there remains a need in the art to develop such treatment regimens, in particular, for elagolix.

### SUMMARY OF THE INVENTION

The disclosure is directed to dosing regimens for GnRH receptor antagonists and, in particular, dosing regimens for 4-((R)-2-[5-(2-fluoro-3-methoxy-phenyl)-3-(2-fluoro-6-trifluoromethyl-benzyl 1)-4-methyl-2,6-dioxo-3,6-dihydro-2H-pyrimidin-1-yl]-1-phenyl-ethylamino)-butyric acid (Compound A) or a pharmaceutically acceptable salt thereof.

Treatment with GnRH receptor antagonists, such as Compound A or pharmaceutically acceptable salt thereof, may be associated with bone mineral density loss. In one aspect, the present disclosure provides dosing regimens for preventing or reducing bone mineral density loss associated with treatment with GnRH receptor antagonists.

In one aspect, this disclosure provides a method for treating a female subject, such as a female subject having endometriosis, adenomyosis, polycystic ovary syndrome (PCOS), or uterine fibroids, with 4-4R)-2-[5-(2-fluoro-3-methoxy-phenyl)-3-(2-fluoro-6-trifluoromethyl-benzyl)-4-methyl-2,6-dioxo-3,6-dihydro-2H-pyrimidin-1-yl]-1-phenyl-ethylamino)-butyric acid (Compound A) or a pharmaceutically acceptable salt thereof, preferably sodium 4-((R)-2-[5-(2-fluoro-3-methoxy-phenyl)-3-(2-fluoro-6-trifluoromethyl-benzyl)-4-methyl-2,6-dioxo-3,6-dihydro-2H-pyri mi din-1-yl]-1-phenyl-ethyl amino) butanoate. The method comprises administering Compound A or a pharmaceutically acceptable salt thereof to the subject according to a first dosing schedule during a first treatment period; and administering Compound A or a pharmaceutically acceptable salt thereof to the subject according to a second dosing schedule during a second treatment period, wherein the second dosing schedule comprises a lower dosage amount, less frequent administration, and/or a lower total daily dose than the first dosing schedule. For example, in a particular embodiment, the first dosing schedule comprises administering about 200 mg sodium 4-4R)-2-[5-(2-fluoro-3-methoxy-phenyl)-3-(2-fluoro-6-trifluoromethyl-benzyl 1)-4-methyl-2,6-dioxo-3,6-dihydro-2H-pyrimidin-1-yl]-1-phenyl-ethylamino) butanoate twice daily to the subject and the second dosing schedule comprises administering about 150 mg sodium 4-((R)-2-[5-(2-fluoro-3-methoxy-phenyl)-3-(2-fluoro-6-trifluoromethyl-benzyl)-4-methyl-2,6-dioxo-3,6-dihydro-2H-pyrimidin-1-yl]-1-phenyl-ethylamino) butanoate once daily to the subject. In certain embodiments, the first treatment period is not more than about six months, such as about three months or about six months. In certain embodiments, the second treatment period is at least six months, such as from about eighteen to about twenty four months.

In one aspect, this disclosure is directed to Compound A or a pharmaceutically acceptable salt thereof, preferably sodium 4-((R)-2-[5-(2-fluoro-3-methoxy-phenyl)-3-(2-fluoro-6-trifluoromethyl-benzyl)-4-methyl-2,6-dioxo-3,6-dihydro-2H-pyrimidin-1-yl]-1-phenyl-ethylamino) butanoate, for use in a method for treating a disease, such as endometriosis, adenomyosis, polycystic ovary syndrome (PCOS), and/or uterine fibroids. In certain embodiments, the method comprises an administration pattern comprising (i) administering Compound A or a pharmaceutically acceptable salt thereof to a subject in need thereof according to a first dosing schedule and (ii) administering Compound A or a pharmaceutically acceptable salt thereof to a subject in need thereof according to a second dosing schedule.

These and other objects of the invention are described in the following paragraphs. These objects should not be deemed to narrow the scope of the invention.

### BRIEF DESCRIPTION OF THE DRAWINGS

FIG. 1 shows observed and model-predicted percentage of subjects with BMD reduction of >5% for the exposure-BMD model at month 6 and month 12.

FIG. 2 shows observed and model-predicted percentage of subjects with BMD reduction of >8% for the exposure-BMD model at month 6 and month 12.

FIG. 3 shows simulated mean % BMD changes and Z-Scores over time for treatment with elagolix 150 mg QD for 24 months.

FIG. 4 shows simulated mean % BMD changes and Z-Scores over time for treatment with elagolix 200 mg BID for 24 months.

FIG. 5 shows simulated BMD changes and Z-Scores over time for elagolix treatment based on DYS response.

FIG. 6 shows simulated BMD changes and Z-Scores over time for elagolix dosing starting with 200 mg BID for 3 months and switching to 150 mg QD up to 24 months.

FIG. 7 shows simulated BMD changes and Z-Scores over time for elagolix dosing starting with 200 mg BID for 6 months and switching to 150 mg QD up to 24 months.

### DETAILED DESCRIPTION OF THE INVENTION

This detailed description is intended only to acquaint others skilled in the art with the present invention, its principles, and its practical application so that others skilled in the art may adapt and apply the invention in its numerous forms, as they may be best suited to the requirements of a particular use. This description and its specific examples are intended for purposes of illustration only. This invention, therefore, is not limited to the embodiments described in this patent application, and may be variously modified.

### A. DEFINITIONS

As used in the specification and the appended claims, unless specified to the contrary, the following terms have the meaning indicated:

The term “APP” as used herein stands for “active pharmaceutical ingredient.” The preferred API as disclosed herein is 4-4R)-2-[5-(2-fluoro-3-methoxy-phenyl)-3-(2-fluoro-6-trifluoromethyl-benzyl)-4-methyl-2,6-dioxo-3,6-dihydro-2H-pyrimidin-1-yl]-1-phenyl-ethylamino)-butyric acid (Compound A) or a pharmaceutically acceptable salt thereof and, preferably is sodium 4-((R)-2-[5-(2-fluoro-3-methoxy-phenyl)-3-(2-fluoro-6-trifluoromethyl-benzyl)-4-methyl-2,6-dioxo-3,6-dihydro-2H-pyrimidin-1-yl]-1-phenyl-ethylamino) butanoate.

As used herein, the term “pharmaceutical composition” means a composition comprising Compound A or a pharmaceutically acceptable salt thereof and, optionally, one or more pharmaceutically acceptable excipients.

The term “pharmaceutically acceptable” is used adjectivally to mean that the modified noun is appropriate for use as a pharmaceutical product for human use or as a part of a pharmaceutical product for human use.

The term “subject” includes humans and other primates as well as other mammals. The term subject includes, for example, a healthy premenopausal female as well as a female patient having, for example, endometriosis, adenomyosis, polycystic ovary syndrome (PCOS), or uterine fibroids. In certain embodiments, the subject is a human. In certain embodiments, the subject is an adult human female. In certain embodiments, the subject is a woman, typically a premenopausal woman, having endometriosis. In certain embodiments, the subject is a woman, typically a premenopausal woman, having adenomyosis. In certain embodiments, the subject is a woman, typically a premenopausal woman, having uterine fibroids.

The term “therapeutically effective amount” means a sufficient amount of the API or pharmaceutical composition to treat a condition, disorder, or disease, at a reasonable benefit/risk ratio applicable to any medical treatment.

The terms “treat”, “treating” and “treatment” refer to a method of alleviating or abrogating a condition, disorder, or disease and/or the attendant symptoms thereof.

### B. DRUG SUBSTANCE

Methods disclosed herein comprise at least one active pharmaceutical ingredient: 4-((R)-2-[5-(2-fluoro-3-methoxy-phenyl)-3-(2-fluoro-6-trifluoromethyl-benzyl 1)-4-methyl-2,6-dioxo-3,6-dihydro-2H-pyrimidin-1-yl]-1-phenyl-ethylamino)-butyric acid (Compound A) or a pharmaceutically acceptable salt thereof.

Compound A has the following formula:

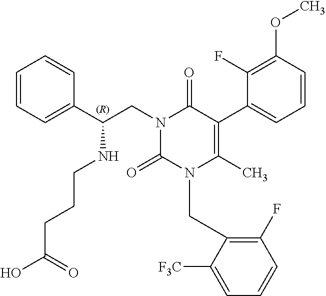

Compound A is an orally active, non-peptide GnRH antagonist and is unlike other GnRH agonists and injectable (peptide) GnRH antagonists. Compound A produces a dose dependent suppression of pituitary and ovarian hormones in women. Methods of making Compound A and a pharmaceutically acceptable salt thereof, as well as similar compounds, are described in WO2001/055119 and WO 2005/007165, the contents of which are herein incorporated by reference.

In certain embodiments, 4-((R)-2-[5-(2-fluoro-3-methoxy-phenyl)-3-(2-fluoro-6-trifluoromethyl-benzyl 1)-4-methyl-2,6-dioxo-3,6-dihydro-2H-pyrimidin-1-yl]-1-phenyl-ethylamino)-butyric acid exists in zwitterionic form. For example, both the carboxylic acid and the tertiary amine are ionized and, thus, the molecule has no overall charge but does have charge separation. Such zwitterionic forms are included within the scope of the term “Compound A or a pharmaceutically acceptable salt thereof.”

Compound A may be administered in the form of acid or base addition salts. Acid addition salts of the free amino compounds of the present invention may be prepared by methods well known in the art, and may be formed from organic and inorganic acids. Suitable organic acids include maleic, fumaric, benzoic, ascorbic, succinic, methanesulfonic, acetic, trifluoroacetic, oxalic, propionic, tartaric, salicylic, citric, gluconic, lactic, mandelic, cinnamic, aspartic, stearic, palmitic, glycolic, glutamic, and benzenesulfonic acids. Suitable inorganic acids include hydrochloric, hydrobromic, sulfuric, phosphoric, and nitric acids. Suitable base addition salts include those salts that form with the carboxyl ate anion and include salts formed with organic and inorganic cations such as those chosen from the alkali and alkaline earth metals (for example, lithium, sodium, potassium, magnesium, barium and calcium), as well as the ammonium ion and substituted derivatives thereof (for example, dibenzylammonium, benzylammonium, 2-hydroxyethylammonium, and the like). Thus, the term “pharmaceutically acceptable salt⁻ of Compound A is intended to encompass any and all acceptable salt forms.

In certain embodiments, Compound A is administered in the form of a pharmaceutically acceptable salt. In certain embodiments, a pharmaceutically acceptable salt of Compound A is the sodium salt of Compound A. The monosodium salt of Compound A has a molecular formula of C32H29F5N3O5Na, which corresponds to a molecular weight of about 653.6 (salt) and about 631.6 (free form). The monosodium salt of Compound A has the following formula:

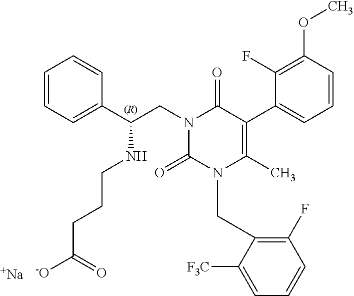

As used herein, unless otherwise noted, amounts in milligrams of Compound A or a pharmaceutically acceptable salt thereof refer to the amount of Compound A free form having a molecular weight of about 631.6. Thus, the term “150 mg” as used herein refers to an amount of a pharmaceutically acceptable salt of Compound A that provides 150 mg of the corresponding free form, such as, for example, about 156 mg of sodium 4-({(1R)-2-[5-(2-fluoro-3-methoxyphenyl)-3-{[2-fluoro-6-(trifluoromethyl)phenyl]methyl]-4-methyl-2,6-dioxo-3,6-dihydropyrimidin-1 (2H)-yl]-1-phenylethyllamino)butanoate. Similarly, the term “200 mg” as used herein refers to an amount of a pharmaceutically acceptable salt of Compound (A) that provides 200 mg of the corresponding free form, such as, for example, about 207 mg of sodium 4-({(1R)-2-[5-(2-fluoro-3-methoxyphenyl)-3-{[2-fluoro-6-(trifluoromethyl)phenyl]methyl}-4-methyl-2,6-dioxo-3,6-dihydropyrimidin-1 (211)-yl]-1-phenyl ethyl}amino) butanoate.

In certain embodiments, Compound A or a pharmaceutically acceptable salt thereof is administered in an amount from about 100 mg to about 350 mg. In some such embodiments, the amount of Compound A, or pharmaceutically acceptable salt thereof, is from about 140 mg to about 160 mg, preferably about 150 mg. In other such embodiments, the amount of Compound A, or pharmaceutically acceptable salt thereof, is from about 190 mg to about 210 mg, preferably about 200 mg.

### C. METHODS OF USE

In one aspect, this disclosure provides a dosing regimen for treating a female subject with 4-((R)-2-[5-(2-fluoro-3-methoxy-phenyl)-3-(2-fluoro-6-trifluoromethyl-benzyl)-4-m ethyl-2,6-di oxo-3,6-dihydro-2H-pyrimidin-1-yl]-1-phenyl-ethyl amino)-butyri c acid (Compound A) or a pharmaceutically acceptable salt thereof, preferably sodium 4-((R)-2-[5-(2-fluoro-3-methoxy-phenyl)-3-(2-fluoro-6-trifluoromethyl-benzyl)-4-methyl-2,6-dioxo-3,6-dihydro-2H-pyrimidin-1-yl|-1-phenyl-ethylamino) butanoate. In certain embodiments, the female subject is suffering from endometriosis, adenomyosis, polycystic ovary syndrome (PCOS), or uterine fibroids. In certain embodiments, the dosing regimen comprises at least two treatment periods—a first treatment period and a second treatment period. Optionally, the dosing regimen may comprise additional treatment periods, such as a third, a fourth, a fifth, or a sixth treatment period, which may sequentially follow the first and second treatment periods or which may be interspersed between the first and second treatment periods.

In certain embodiments, the first treatment period comprises a first dosing schedule. In some such embodiments, the first dosing schedule comprises a dosage amount, such as for example, 150 mg or 200 mg. In some such embodiments, the first dosing schedule comprises a dosing frequency, such as for example, once per day (“QD”) or twice per day (“BID”). In a particular embodiment, the first dosing schedule may comprise administration of 200 mg of Compound A or pharmaceutically acceptable salt thereof twice daily. In another particular embodiment, the first dosing schedule may comprise administration of 150 mg of Compound A or pharmaceutically acceptable salt thereof once daily.

In certain embodiments, the first treatment period lasts for at least two weeks or, alternatively, at least one month. In certain embodiments, the first treatment period lasts for not more than about six months. In some such embodiments, the first treatment period is about one, about two, about three, about four, about five, or about six months. In some such embodiments, the first treatment period is from about three months to about six months.

In certain embodiments, the second treatment period comprises a second dosing schedule. In some such embodiments, the second dosing schedule comprises a dosage amount, such as for example, 150 mg or 200 mg. In some such embodiments, the second dosing schedule comprises a dosing frequency, such as for example, QD or BID. In a particular embodiment, the second dosing schedule may comprise administration of 150 mg of Compound A or pharmaceutically acceptable salt thereof once daily. In another particular embodiment, the second dosing schedule may comprise administration of 200 mg of Compound A or pharmaceutically acceptable salt thereof twice daily.

In certain embodiments, the second treatment period lasts for at least three months or, alternatively, at least six months, or alternatively at least one year. In some such embodiments, the first treatment period is about six, about seven, about eight, about nine, about ten, about eleven, about twelve, about thirteen, about fourteen, about fifteen, about sixteen, about seventeen, about eighteen, about nineteen, about twenty, or about twenty one months. In some such embodiments, the second treatment period is from about eighteen months to about twenty one months.

In certain embodiments, the first and second treatment periods are separated by another treatment period or a non-treatment period. In certain embodiments, second treatment period begins immediately following the first treatment period.

In certain embodiments, the dosing regimen further comprises an observation period. In some such embodiments, the observation period comprises an assessment of one or more disease symptoms or efficacy parameters, such as dysmenorrhea or non-menstrual pelvic pain, and/or one or more potential side effects or safety parameters, such bone mineral density. In some such embodiments, Compound A or pharmaceutically acceptable salt thereof is not administered to the subject during the observation period. In other such embodiments, Compound A or pharmaceutically acceptable salt thereof is administered to the subject during the observation period. In certain embodiments, the observation period overlaps with the first treatment period and/or the second treatment period. In some such embodiments, the observation period overlaps with the first treatment period. In some such embodiments, the observation period overlaps with the second treatment period. In some such embodiments, the observation period overlaps with the first treatment period and the second treatment period. In certain embodiments, the observation period does not overlap with the first treatment period or the second treatment period. In some such embodiments, the observation period does not overlap with the first treatment period. In some such embodiments, the observation period does not overlap with the second treatment period. In some such embodiments, the observation period does not overlap with any treatment period.

In certain embodiments, the first treatment period ends if the subject is determined to be a non-responder to administration of Compound A or a pharmaceutically acceptable salt thereof according to the first dosing schedule. In certain other embodiments, the first treatment period ends after disappearance of one or more symptoms of the disease.

In certain embodiments, the first treatment period ends if the subject is determined to be experiencing an unacceptable side effect. In certain other embodiments, the first treatment period ends upon development or appearance of an unacceptable side effect.

In one aspect, this disclosure provides a method for treating a female subject with 4-4R)-2-[5-(2-fluoro-3-methoxy-phenyl)-3-(2-fluoro-6-trifluoromethyl-benzyl)-4-methyl-2,6-dioxo-3,6-dihydro-2H-pyrimidin-1-yl]-1-phenyl-ethylamino)-butyric acid (Compound A) or a pharmaceutically acceptable salt thereof, preferably sodium 4-((R)-2-[5-(2-fluoro-3-methoxy-phenyl)-3-(2-fluoro-6-trifluoromethyl-benzyl)-4-methyl-2,6-dioxo-3,6-dihydro-2H-pyrimidin-1-yl]-1-phenyl-ethylamino) butanoate. The method comprises administering Compound A or a pharmaceutically acceptable salt thereof to the subject according to a first dosing schedule during a first treatment period; and administering Compound A or a pharmaceutically acceptable salt thereof to the subject according to a second dosing schedule during a second treatment period, wherein the second dosing schedule comprises a lower dose and/or less frequent administration than the first dosing schedule. In certain embodiments, the first treatment period is not more than about six months, such as about three months or about six months. In certain embodiments, the second treatment period is from about eighteen to about twenty four months. In certain embodiments, the subject is suffering from endometriosis, adenomyosis, polycystic ovary syndrome (PCOS), or uterine fibroids.

In certain embodiments, Compound A or a pharmaceutically acceptable salt thereof is administered at a specified interval during the first treatment period. In some such embodiments, the specified interval during the first treatment period is once per day. In some such embodiments, the specified interval during the first treatment period is twice per day.

In certain embodiments, Compound A or a pharmaceutically acceptable salt thereof is administered at a specified dosage amount during the first treatment period. In some such embodiments, the specified dosage amount during the first treatment period is 150 mg. In some such embodiments, the specified dosage amount during the first treatment period is 200 mg.

In certain embodiments, Compound A or a pharmaceutically acceptable salt thereof is administered at a specified total daily dose during the first treatment period. In some such embodiments, the specified total daily dose during the first treatment period is 150 mg per day. In some such embodiments, the specified total daily dose during the first treatment period is 400 mg per day.

In certain embodiments, Compound A or a pharmaceutically acceptable salt thereof is administered at a specified interval during the second treatment period. In some such embodiments, the specified interval during the second treatment period is once per day. In some such embodiments, the specified interval during the second treatment period is twice per day.

In certain embodiments, Compound A or a pharmaceutically acceptable salt thereof is administered at a specified dosage amount during the second treatment period. In some such embodiments, the specified dosage amount during the second treatment period is 150 mg. In some such embodiments, the specified dosage amount during the second treatment period is 200 mg.

In certain embodiments, Compound A or a pharmaceutically acceptable salt thereof is administered at a specified total daily dose during the second treatment period. In some such embodiments, the specified total daily dose during the second treatment period is 150 mg per day. In some such embodiments, the specified total daily dose during the second treatment period is 400 mg per day.

In certain particular embodiments, the first dosing schedule comprises twice daily administration of about 200 mg Compound A or a pharmaceutically acceptable salt thereof. In certain particular embodiments, the second dosing schedule comprises once daily administration of about 150 mg Compound A or a pharmaceutically acceptable salt thereof

In one aspect, this disclosure provides a method reducing the rate of bone mineral density loss in a subject treated with 4-((R)-2-[5-(2-fluoro-3-methoxy-phenyl)-3-(2-fluoro-6-trifluoromethyl-benzyl)-4-methyl-2,6-dioxo-3,6-dihydro-2H-pyrimidin-1-yl]-1-phenyl-ethylamino)-butyric acid (Compound A) or a pharmaceutically acceptable salt thereof, preferably sodium 4-4R)-2-[5-(2-fluoro-3-methoxy-phenyl)-3-(2-fluoro-6-trifluoromethyl-benzyl)-4-methyl-2,6-dioxo-3,6-dihydro-2H-pyrimidin-1-yl]-1-phenyl-ethylamino) butanoate. The method comprises administering Compound A or a pharmaceutically acceptable salt thereof to the subject according to a first dosing schedule during a first treatment period, wherein the first dosing schedule is associated with a first rate of bone mineral density loss. The method further comprises, administering Compound A or a pharmaceutically acceptable salt thereof to the subject according to a second dosing schedule during a second treatment period, wherein the second dosing schedule is associated with a second rate of bone mineral density loss that is reduced relative to the first rate of bone mineral density loss.

In certain embodiments, the second dosing schedule comprises a reduced dosage amount, dosing frequency, and/or total daily dose relative to the first dosing schedule. In certain embodiments, the first treatment period is not more than about six months, such as about three months or about six months. In certain embodiments, the second treatment period is from about eighteen to about twenty four months. In certain embodiments, the subject is suffering from endometriosis, adenomyosis, polycystic ovary syndrome (PCOS), or uterine fibroids.

In at least one aspect, this disclosure provides a method of treating endometriosis in a subject in need thereof comprising administering about 200 mg sodium 4-((R)-2-[5-(2-fluoro-3-methoxy-phenyl)-3-(2-fluoro-6-trifluoromethyl-benzyl 1)-4-methyl-2,6-dioxo-3,6-dihydro-2H-pyrimidin-1-yl]-1-phenyl-ethylamino) butanoate twice daily to the subject for up to six months and subsequently administering about 150 mg sodium 4-((R)-2-[5-(2-fluoro-3-methoxy-phenyl)-3-(2-fluoro-6-trifluoromethyl-benzyl)-4-methyl-2,6-dioxo-3,6-dihydro-2H-pyrimidin-1-yl]-1-phenyl-ethylamino) butanoate once daily to the subject.

In at least one aspect, this disclosure provides a method of treating adenomyosis in a subject in need thereof comprising administering about 200 mg sodium 4-((R)-2-[5-(2-fluoro-3-methoxy-phenyl)-3-(2-fluoro-6-trifluoromethyl-benzyl 1)-4-methyl-2,6-dioxo-3,6-dihydro-2H-pyrimidin-1-yl]-1-phenyl-ethylamino) butanoate twice daily to the subject for up to six months and subsequently administering about 150 mg sodium 4-((R)-2-[5-(2-fluoro-3-methoxy-phenyl)-3-(2-fluoro-6-trifluoromethyl-benzyl)-4-methyl-2,6-dioxo-3,6-dihydro-2H-pyrimidin-1-yl]-1-phenyl-ethylamino) butanoate once daily to the subject.

In at least one aspect, this disclosure provides a method of treating uterine fibroids in a subject in need thereof comprising administering about 200 mg sodium 4-((R)-2-[5-(2-fluoro-3-methoxy-phenyl)-3-(2-fluoro-6-trifluoromethyl-benzyl 1)-4-methyl-2,6-dioxo-3,6-dihydro-2H-pyrimidin-1-yl]-1-phenyl-ethylamino) butanoate twice daily to the subject for up to six months and subsequently administering about 150 mg sodium 4-((R)-2-15-(2-fluoro-3-methoxy-phenyl)-3-(2-fluoro-6-trifluoromethyl-benzyl)-4-methyl-2,6-dioxo-3,6-dihydro-2H-pyrimidin-1-yl]-1-phenyl-ethylamino) butanoate once daily to the subject.

In at least one aspect, this disclosure provides a method of treating uterine fibroids in a subject in need thereof comprising administering about 300 mg sodium 4-((R)-2-[5-(2-fluoro-3-methoxy-phenyl)-3-(2-fluoro-6-trifluoromethyl-benzyl 1)-4-methyl-2,6-dioxo-3,6-dihydro-2H-pyrimidin-1-yl]-1-phenyl-ethylamino) butanoate twice daily to the subject for a treatment period of more than six months and co-administering a hormone add-back therapy during the treatment period. In certain embodiments, the hormone add-back therapy comprises estradiol and norethindrone acetate.

In one aspect, this disclosure provides a method and a dosing regimen for treating a female subject with endometriosis, adenomyosis, polycystic ovary syndrome (PCOS), or uterine fibroids with 4-((R)-2-[5-(2-fluoro-3-methoxy-phenyl)-3-(2-fluoro-6-trifluoromethyl-benzyl)-4-methyl-2,6-dioxo-3,6-dihydro-2H-pyrimidin-1-yl]-1-phenyl-ethylamino)-butyric acid (Compound A) or a pharmaceutically acceptable salt thereof, preferably sodium 4-((R)-2-[5-(2-fluoro-3-methoxy-phenyl)-3-(2-fluoro-6-trifluoromethyl-benzyl)-4-methyl-2,6-dioxo-3,6-dihydro-2H-pyrimidin-1-yl]-1-phenyl-ethylamino) butanoate for a treatment period of more than six months, alternatively up to twelve months, or alternatively more than twelve months. The method and dosing regimen comprises co-administration of a hormone add-back therapy during the treatment period. In a particular embodiment, the female subject has heavy menstrual bleeding (HMB) associated with uterine fibroids.

In certain embodiments, the treatment period lasts for more than six months. In certain embodiments, the treatment period lasts for up to twelve months. In some such embodiments, the treatment period is about seven, about eight, about nine, about ten, about eleven, or about twelve months. In some such embodiments, the treatment period is from about nine months to about twelve months. In certain embodiments, the treatment period lasts for more than twelve months.

In certain embodiments, Compound A or pharmaceutically acceptable salt thereof is administered according to an elagolix dosing schedule. In some such embodiments, the elagolix dosing schedule comprises an elagolix dosage amount, such as for example, 150 mg, 200 mg, or 300 mg. In some such embodiments, the elagolix dosing schedule comprises an elagolix dosing frequency, such as for example, once per day (“QD”) or twice per day (“BID”). In a particular embodiment, the elagolix dosing schedule may comprise administration of 300 mg of Compound A or pharmaceutically acceptable salt thereof twice daily.

In certain embodiments, the hormone add-back therapy comprises a progestogen, such as a progestin, and an estrogen. In some such embodiments, the progestogen is norethindrone or norethindrone acetate. In some such embodiments, the estrogen is estradiol.

In certain embodiments, the hormone add-back therapy is administered according to an add-back dosing schedule. In some such embodiments, the add-back dosing schedule comprises a progestogen dosage amount, such as for example, 0.1 mg or 0.5 mg norethindrone acetate. In some such embodiments, the add-back dosing schedule comprises a progestogen dosing frequency, such as for example, once per day (“QD”). In some such embodiments, the add-back dosing schedule comprises an estrogen dosage amount, such as for example, 0.5 mg or 1.0 mg estradiol. In some such embodiments, the add-back dosing schedule comprises an estrogen dosing frequency, such as for example, once per day (“QD”). In a particular embodiment, the add-back dosing schedule may comprise administration of 1.0 mg estradiol and 0.5 mg norethindrone acetate once daily.

In certain embodiments, any of the above methods further comprise administering to the subject a hormone to reduce or alleviate potential side effects of Compound A or a pharmaceutically acceptable salt thereof. For example, the method may comprise administration of an estrogen, a progestin, or a combination thereof. Such treatments are commonly referred to as “add-back” therapy.

In some such embodiments, the add-back therapy comprises a progestogen, such as a progestin. In some such embodiments, the add-back therapy comprises an estrogen. In some such embodiments, the add-back therapy comprises a progestin and an estrogen.

The estrogen and/or progestogen can be administered orally, transdermally or intravaginally. Suitable progestogens for use in the add-back therapy include, for example, progesterone, norethindrone, norethindrone acetate, norgestimate, drospirenone, and medroxyprogestogen. Suitable estrogens for use in the add-back therapy include, for example, estradiol, ethinyl estradiol, and conjugated estrogens. Combined oral formulations containing an estrogen and a progestogen are known in the art and include, for example, Activella®, Angeliq®, FemHRT®, Jenteli™, Mimvey™, Prefest™, Premphase®, and Prempro®.

In certain embodiments, the estrogen is estradiol, ethinyl estradiol, or a conjugated estrogen. In some such embodiments, the estrogen is estradiol. In some such embodiments, the estradiol is administered once a day. In some such embodiments, the dose of estradiol is 0.5 mg. In other such embodiments, the dose of estradiol is 1.0 mg. In some such embodiments, the estrogen is ethinyl estradiol. In some such embodiments, the ethinyl estradiol is administered once a day. In some such embodiments, the dose of ethinyl estradiol is 2.5 meg. In other such embodiments, the dose of ethinyl estradiol is 5.0 meg. In some such embodiments, the estrogen is a conjugated estrogen. In some such embodiments, the conjugated estrogen is administered once a day. In some such embodiments, the dose of conjugated estrogen is 0.3 mg. In other such embodiments, the dose of conjugated estrogen is 0.45 mg or 0.625 mg.

In certain embodiments, the progestogen is progesterone, norethindrone, norethindrone acetate, norgestimate, medroxyprogesterone, or drospirenone. In some such embodiments, the progestogen is oral progesterone. In some such embodiments, the oral progesterone is used cyclically (for the last 12 days of the 28-30 day cycle). In some such embodiments, the dose of the oral progesterone is 100 or 200 mg. In some such embodiments, the progestogen is norethindrone or norethindrone acetate. In some such embodiments, the norethindrone or norethindrone acetate is administered once a day. In some such embodiments, the dose of norethindrone or norethindrone acetate is 0.1 mg. In some such embodiments, the dose of norethindrone or norethindrone acetate is 0.5 mg. In some such embodiments, the dose of norethindrone or norethindrone acetate is 1.0 mg. In some such embodiments, the progestogen is norgestimate. In some such embodiments, the norgestimate is administered once a day. In some such embodiments, the dose of norgestimate is 0.09 mg. In some such embodiments, the progestogen is medroxyprogesterone. In some such embodiments, the medroxyprogesterone is administered once a day. In some such embodiments, the dose of medroxyprogesterone is 1.5 mg. In some such embodiments, the dose of medroxyprogesterone is 2.5 mg or 5 mg. In some such embodiments, the progestogen is drospirenone. In some such embodiments, the drospirenone is administered once a day. In some such embodiments, the dose of drospirenone is 0.25 mg. In some such embodiments, the dose of drospirenone is 0.5 mg.

In certain embodiments, the add-back therapy comprises a norethisterone prodrug, such as norethindrone acetate. In some such embodiments, the add-back therapy further comprises estradiol. Thus, in some such embodiments, the add-back therapy comprises estradiol and norethindrone acetate. In some such embodiments, estradiol and norethindrone acetate are administered orally once per day. In some such embodiments, estradiol is administered in an amount of about 0.5 mg and norethindrone acetate is administered in an amount of about 0.1 mg per day. In other such embodiments, estradiol is administered in an amount of about 1.0 mg and norethindrone acetate is administered in an amount of about 0.5 mg per day. Alternatively, in certain embodiments, estradiol is administered continuously and norethindrone acetate is administered once per day during the last 12-14 days of a menstrual cycle.

In certain embodiments, the dose of Compound A or a pharmaceutically acceptable salt thereof is administered twice a day. In some such embodiments, add-back therapy is administered once a day. The administration of Compound A or a pharmaceutically acceptable salt thereof may be prior to, immediately prior to, during, immediately subsequent to or subsequent to the administration of the add-back therapy.

In certain embodiments, Compound A or pharmaceutically acceptable salt thereof is administered according to an elagolix dosing schedule, such as about 300 mg twice daily and an add-back therapy, such as a combination of an estrogen and a progestogen (e.g., estradiol and norethindrone acetate) is administered according an add-back dosing schedule, such as estradiol 1.0 mg/norethindrone acetate 0.5 mg once daily.

In certain embodiments, a dose of Compound A or pharmaceutically acceptable salt thereof (e.g., 200 mg) is administered in the morning with add-back therapy, such as a combination of an estrogen and a progestogen (e.g., estradiol and norethindrone acetate) and a dose of Compound A or pharmaceutically acceptable salt thereof (e.g., 200 mg) is administered in the evening without add-back therapy.

In certain embodiments, Compound A or a pharmaceutically acceptable salt thereof is co-packaged with the add-back therapy. For example, a blister pack may contain a dose of Compound A or a pharmaceutically acceptable salt thereof and a dose of the add-back therapy.

In certain embodiments, Compound A or a pharmaceutically acceptable salt thereof is present in a fixed dose combination with the add-back therapy. For example, a capsule may contain a caplet or tablet comprising Compound A or a pharmaceutically acceptable salt thereof and a caplet or tablet comprising the add-back therapy, such as a combination of an estrogen and a progestogen (e.g., estradiol and norethindrone acetate). In some such embodiments, the capsule comprises 200 mg Compound A or a pharmaceutically acceptable salt thereof, 1 mg estradiol, and 0.5 mg norethindrone acetate. In some such embodiments, the capsule comprises 300 mg Compound A or a pharmaceutically acceptable salt thereof, 1 mg estradiol, and 0.5 mg norethindrone acetate.

### D. EXAMPLES

The following Examples demonstrate certain challenges encountered during formulation development and describe formulations that overcome those challenges.

### Example 1: Exposure-BMD Modeling

An exposure-BMD model was built to describe the relationship between elagolix exposure and changes in lumbar spine BMD and to predict BMD changes following different elagolix treatment regimens. This model reasonably predicted the BMD changes observed in Phase 3 clinical trials.

Model development was based on 12 months lumbar spine BMD data from 2 endometriosis pivotal randomized controlled studies and 2 uncontrolled extension studies. Simulations of elagolix effects on BMD beyond 12 months are extrapolated.

The exposure-BMD model was built using nonlinear mixed effects modeling in NONMEM 7.3 (model analysis program) compiled with the GNU Fortran compiler (Version 4.8.3). The infrastructure for model development and evaluation of the final model was a cluster featuring 42 Hewlett-Packard ProLiant servers under the OpenSUSE operating system with MOSIX Cluster and Grid Management (Version 4.4.0). NONMEM was used for the BMD safety analysis, where the BMD model parameters were estimated using the first-order conditional estimation method with mg INTERACTION (FOCEI) as implemented in NONMEM.

Exposure-BMD modeling was conducted in a step-wise manner: first developing the appropriate structural model with using the appropriate residual error model, followed by models for interindividual variability, and then testing of potential covariates.

The model was conceptualized as an indirect response model that describes the change from baseline BMD (BLBMD) and assumes a baseline steady state between bone formation and resorption as follows:

$$\begin{matrix}\frac{dR ( t )}{dt} = {K}_{in} - {K}_{out} * R ( t ) & Equation 1 \\ BMD ( t ) = BLBMD * R ( t ) & Equation 2\end{matrix}$$

and at baseline:

$$R ( 0 ) = 1 and {K}_{out} = {K}_{in} / 1 \qquad Equation 3$$

where dR(t)/dt is the change in BMD over time, Km is a zero-order rate constant reflecting bone formation, Kₒᵤₜ is a first-order rate constant reflecting bone resorption, BMD (t) is the BMD at time (t), and R (t) is the change in BMD from baseline (BLBMD) at time (t).

Baseline BMD was modelled as a typical value for the population with its associated interindividual variability, and a different baseline value was estimated for each type of DXA scan machine (Hologic and Lunar) used.

$$BLBMDi = TVBLBMD * {exp}^{η i} \qquad Equation 4$$

In order to characterize changes in BMD in women treated with placebo, a placebo model was first developed using data from the placebo arms in two randomized, double-blind, placebo-controlled studies designed to evaluate elagolix in women with moderate-to-severe endometriosis-associated pain.

First, a model that assumes no change in BMD from baseline was fitted to the placebo data.

$$\frac{dR ( t )}{dt} = 0 \qquad Equation 5$$

The model was then compared to a model including BMD change in subjects on placebo described by the parameter (PLAC_EFF) to reflect changes not related to elagolix

$$\frac{dR ( t )}{dt} = PLAC\_EFF \qquad Equation 6$$

treatment.

Once the placebo model that best describes observed BMD changes in the placebo arm was selected, the exposure-BMD model was then built after fixing the placebo effect relevant parameters by adding the response to elagolix treatment via an indirect response model using data from the active treatment arms from 2 endometriosis pivotal randomized controlled studies and 2 uncontrolled extension studies. Individual monthly average elagolix concentrations (Cavg) derived from the final population pharmacokinetic analysis were used as exposure measures in the exposure-BMD model. Preliminary exposure-BMD regression analyses demonstrated that elagolix average concentrations are better predictors of BMD changes compared to trough concentrations.

The effects of elagolix on BMD were modeled using a stimulatory Emax function on the bone resorption (Kout), as follows:

$$\frac{dR ( t )}{dt} = {k}_{in} - {k}_{out} \cdot ( 1 - \frac{{E}_{max} \cdot {C}_{avg}^{HILL}}{{EC}_{50}^{HILL} + {C}_{avg}^{HILL}} ) \cdot R ( t ) + PLAC\_EFF \qquad Equation 7$$

where, Eₘₐₓ is the elagolix maximum stimulatory effect on Kₒᵤₜ, EC₅₀ is the elagolix monthly average concentration producing 50% of maximum stimulation, and HILL is the stimulatory Eₘₐₓ curve shape factor.

Interindividual variabilities (ITV) in model parameters were modeled using a log normal distribution, and were only included if a significant improvement of the model fit was achieved (p<0.01) and model stability was maintained.

Covariate effects were then included into the model in a multiplicative fashion. Continuous covariates, except the screening Z-score, were normalized to a reference value (median value of the study population) and included in the model with a power function. The screening Z-score was tested as a linear instead of a power model since negative values can be observed. Dichotomous categorical covariates were tested with a multiplicative model in order to obtain the fractional difference of the parameters between the tested categorical groups. Inferences about the clinical importance of covariate effects were made based on the magnitude and precision of covariate parameter estimates.

Finally, individual model parameters were modeled as follows:

$${θ}_{i , k} = {θ}_{k} \cdot ( {\prod }_{p = 1}^{{n}_{p}} {( \frac{{cov}_{i , p}}{{ref}_{p}} )}^{{n}_{k , p}} \cdot {\prod }_{q = 1}^{{n}_{q}} ( 1 + {θ}_{k , q} ( {cov}_{i , q} ) \cdot ( 1 + {θ}_{sensor} * ( SCZSCOR - median ( SCZSCOR ) ) \cdot {e}^{( {η}_{ik} )} \qquad Equation 8$$

where θ_{i,k} is the value of the k^{th} parameter in the i^{th} subject, θₖ is the typical value of the k^{th} parameter, nₚ is the number of continuous covariates, cov_{i,p} is the p^{th} continuous covariate value in the i^{th} subject, refₚ is the median values for the p^{th} continuous covariate, θ_{k,p} is the p^{th} continuous covariate parameter estimate for the k^{th} parameter, n_{q} is the number of dichotomous covariates, θ_{k,q} is the q^{th} categorical covariate parameter estimate for the fractional change of the k^{th} parameter, cov_{i,q} is the q^{th} categorical covariate indicator value (0 or 1) for the i^{th} subject, and n_{i,k} is the individual-specific random effects for the k^{th} parameter in the i^{th} subject. The n_{i,k} values are assumed to be multivariate normally distributed: η˜N(0, ω²), with mean vector 0 and variance elements denoted by ω²ₖ for the k^{th} parameter.

Residual variability was modeled using the additive, proportional (constant coefficient of variation), or a combination of additive and proportional error models as follows:

$$\begin{matrix}{BMD}_{ij} = + {ε}_{ij} & Equation 9 \\ {BMD}_{ij} = * ( 1 + {ε}_{ij} ) & Equation 10 \\ {BMD}_{ij} = * ( 1 + {ε}_{1 ij} ) + {ε}_{2 ij} & Equation 11\end{matrix}$$

where BMDᵢⱼ is the j^{th} observed BMD measurement in individual i, BMDᵢⱼ is the j^{th} model-predicted BMD value in individual i, and ϵᵢⱼ is the residual random error for individual i and measurement j. The E values were assumed to be independently and identically distributed with a means of 0 and variances of σ²:ϵ˜N(0, σ²).

Relevant covariate-parameter relationships were investigated using forward inclusion and backward elimination procedures. Covariates were tested on parameters where interindividual variability had been identified. The covariates tested in this analysis included: demographics (age, weight, body mass index [BMI], race, tobacco use, alcohol use, region (outside of the United States [non-US] versus [US]); hormone (E2, progesterone, luteinizing hormone [LH] and follicle stimulating hormone [FSH] concentration at baseline); screening Z-score (which corrects for differences due to different Dual Energy X-Ray Absorptiometry [DXA] scanning machines); baseline bone biomarkers (osteocalcin, type I collagen C-telopeptide [CTX], and procollagen type I N-propeptide [PINP]) concentrations; concomitant medication (calcium use and vitamin D use); and prior GnRH therapy.

### Goodness of Fit Plots for Final Exposure—BMD Model

During the model development process, graphical methods were employed to assess model goodness-of-fit. Goodness-of-fit plots were generated ad hoc for model evaluation and included: observed versus predicted plots comparing population and individual predictions to observations in separate plots; conditional weighted residuals (CWRES) plotted versus population predictions and versus time; histograms and QQ plots of interindividual random effects (ETAs) and CWRES; and scatter plots of the random effect correlation matrix.

The goodness-of-fit for the exposure-BMD model was evaluated graphically. The plots of predicted and observed BMD indicated that the model adequately described the observations over the entire range. It is noteworthy that since two separate BLBMD values are estimated depending on the machine used for DXA scanning, two clusters of PREDs were observed in the PRED versus observed BMD (DV) plot. CWRES did not show any major trends when plotted against sampling time or population predictions, indicating that the model was appropriately unbiased.

### Simulation-Based Model Evaluation

The exposure-BMD model was used to run 1000 simulated replicates of the dataset using NONMEM $SIMULATION option. The final simulation results were used to predict percentages of subjects exceeding specific thresholds of BMD change (i.e., <˜3%, <5%, and <−8%) under each dosing regimen; and were compared against the observed percentages.

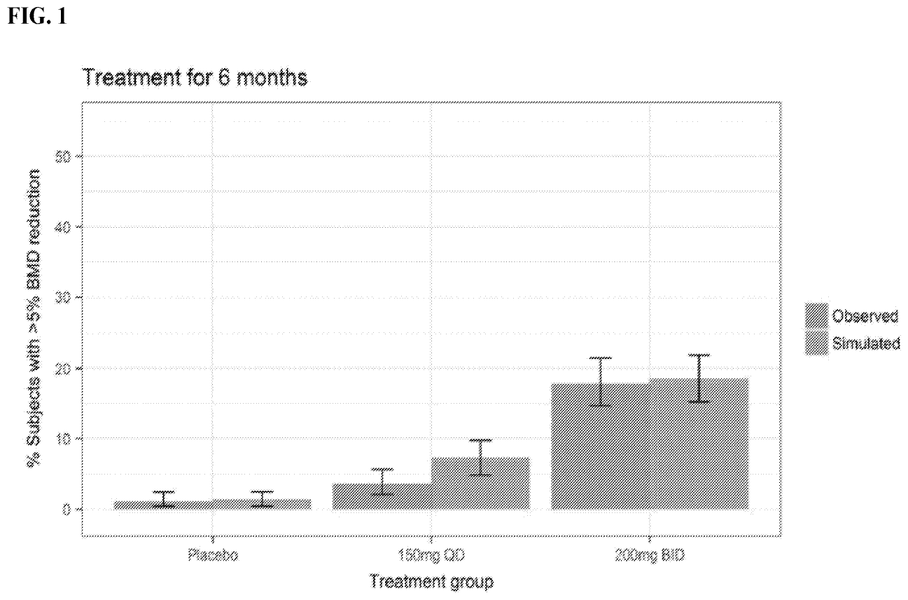

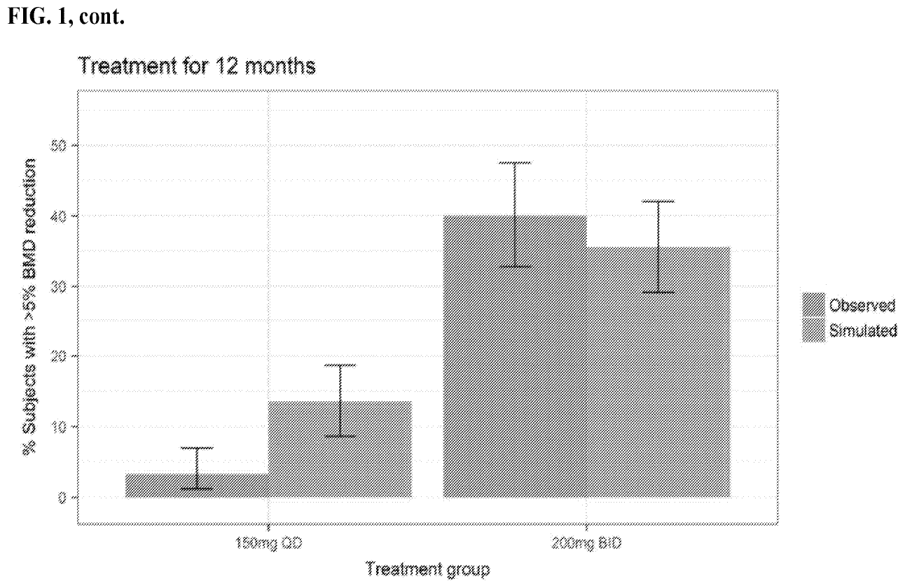

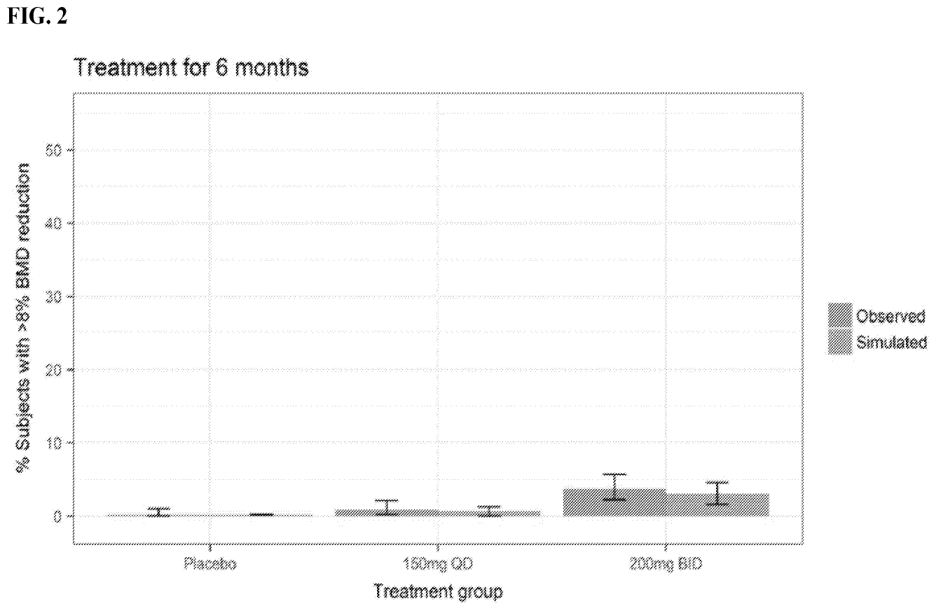

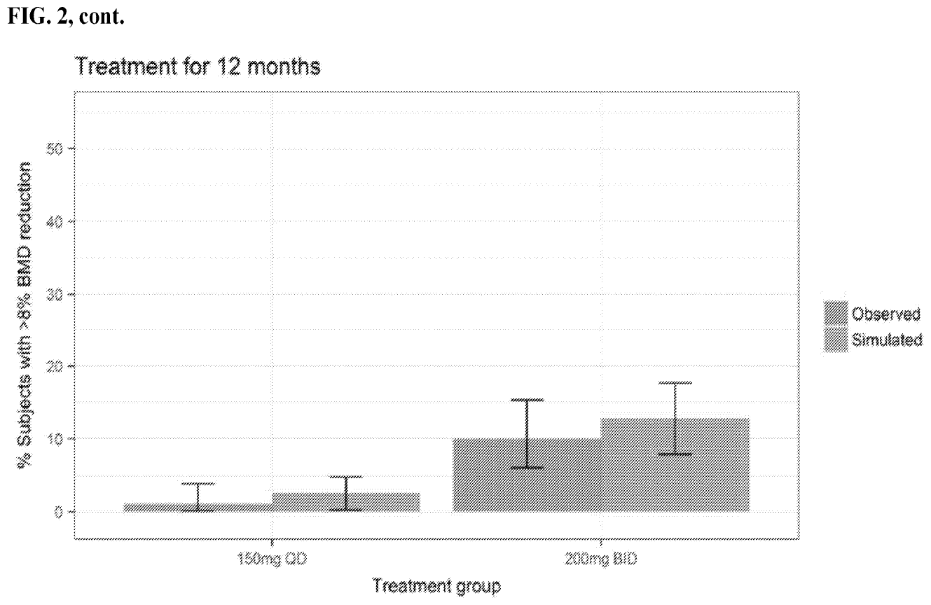

A comparison of the observed and model predicted percentage of subjects with bone loss >−5% and >−8% are shown FIG. 1 and FIG. 2, respectively. Overall, the percentage of subjects with bone loss above certain thresholds was captured reasonably well by the exposure-BMD model across the range of doses included in the analysis.

### Simulations for Continuous Dosing for 24 Months

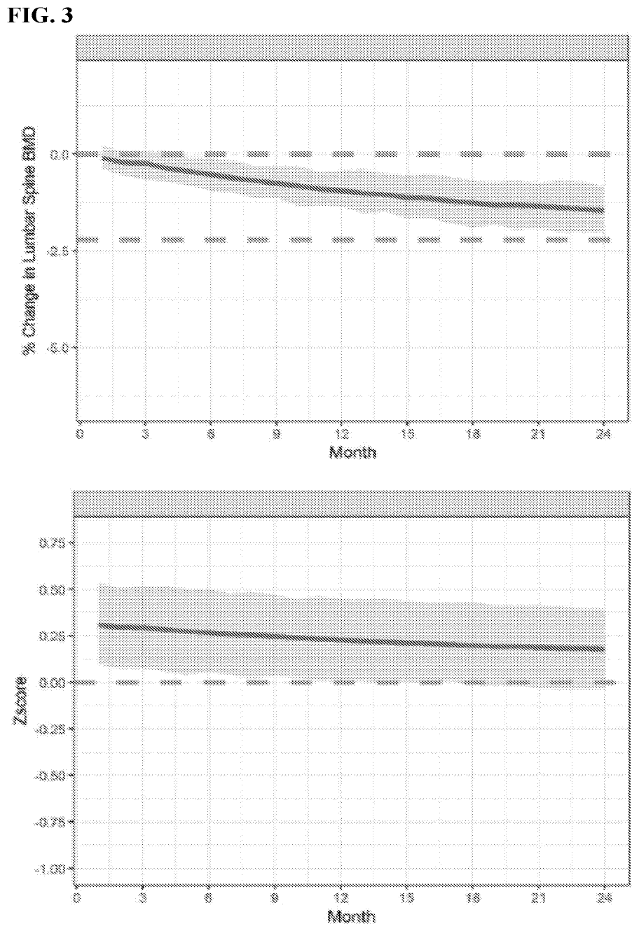

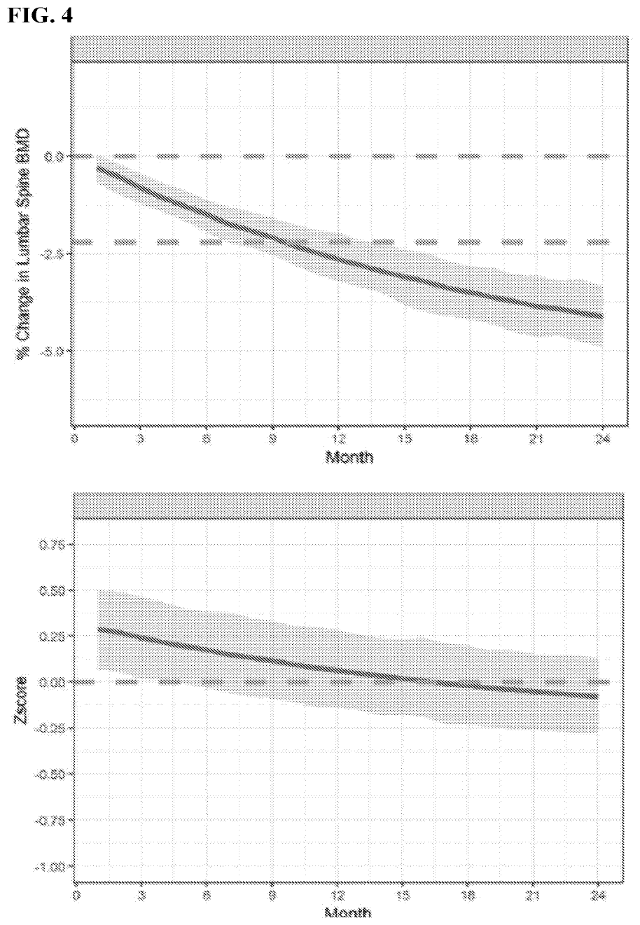

In this scenario each simulated subject was treated with 150 mg QD or 200 mg BID for 24 months and the % BMD change from baseline as well as Z-score were predicted over the treatment period. In FIG. 3, the mean % change in BMD and the mean Z-score over time together with 95% confidence intervals under the 70% compliance assumption for 150 mg QD are shown. Summary statistics for BMD % change and Z-score at months 12 and 24 are shown in Table 1 together with % of subjects with a Z-score <−1.5 or −2 at each time point. Corresponding results for treatment with 200 mg BID are shown in FIG. 4 and Table 2.

TABLE 1
Summary Statistics of Predicted Mean % BMD Changes and Z-Scores
for Treatment with Elagolix 150 mg QD for 24 Months.

| | Mean % | | Mean | | | |
|---|---|---|---|---|---|---|
| | Change | | Z-Score | | % subjects | % subjects |
| Month | in BMD | 95% CI | (ZS) | 95% CI | ZS < −1.5 | ZS < −2.0 |
| 6 | −0.519 | −0.955-−0.0885 | 0.269 | 0.0594-0.502 | 4.56 | 1.44 |
| 12 | −0.942 | −1.34-−0.408 | 0.228 | 0.0120-0.448 | 5.09 | 1.57 |
| 24 | −1.45 | −2.04-−0.814 | 0.179 | 0.0352-0.401 | 5.75 | 1.96 |

TABLE 2
Summary Statistics of Predicted Mean % BMD Changes and Z-Scores
for Treatment with Elagolix 200 mg QD for 24 Months.

| | Mean % | | Mean | | | |
|---|---|---|---|---|---|---|
| | Change | | Z-Score | | % subjects | % subjects |
| Month | in BMD | 95% CI | (ZS) | 95% CI | ZS < −1.5 | ZS < −2.0 |
| 6 | −1.48 | −1.95-−1.12 | 0.176 | −0.0353-0.387 | 5.49 | 1.75 |
| 12 | −2.65 | −3.21-−1.96 | 0.0628 | −0.138-0.282 | 7.02 | 2.38 |
| 24 | −4.12 | −4.88-−3.32 | −0.0804 | −0.282-0.129 | 9.87 | 3.44 |

### Results and Summary

Exposure-BMD modeling using data from four Phase 3 studies in women with endometriosis-associated pain revealed an exposure-response relationship between elagolix average concentrations and changes in BMD. The estimated ECso of 240 ng/ml is more than 5-fold of the predicted exposure with 150 mg QD dosing (Cave concentrations of −47 ng/ml). Such a difference is reflected in the small BMD change with 150 mg QD dosing (−1% BMD change from baseline after 12 months) and suggests that clinically relevant BMD changes may not be expected in most women treated with the 150 mg QD dose of elagolix.

Model results showed that subject race, baseline BMI and CTX levels (a bone resorption biomarker) were significant predictors of baseline BMD. Specifically, African-American race, higher BMI, and lower CTX levels were all associated with higher baseline BMD. In addition to its effects on baseline BMD, BMI was also significantly associated with higher bone formation rates (K).

After incorporating the above covariates, none of the tested covariates (including baseline BMD, expressed as Z-score) were significantly associated with BMD changes due to elagolix treatment (i.e., Emax and EC5o).

Simulations of elagolix 150 mg QD dosing up to 24 months using the model showed that the predicted mean % change from baseline BMD is −1.5% with 2% of subjects predicted to have a Z score of <−2. Similarly, simulations for continuous dosing with elagolix 200 mg BID for 24 months showed that 200 mg BID dosing is predicted to result in BMD changes of −2.6% and −4.1% after 12 and 24 months of treatment, respectively. Such changes were predicted to result in approximately 2% and 3% of women with a Z-score of <−2 following 12 and 24 months of treatment, respectively. Such results indicate that a large proportion of women treated with elagolix 150 mg QD or 200 mg BID may not experience clinically relevant changes in Z-score and hence, may benefit from long term treatment with elagolix with appropriate BMD monitoring.

The exposure-BMD indirect-response model with zero-order bone formation and first-order bone resorption rates, adequately predicted the observed BMD changes during treatment and follow-up periods of the Phase 3 studies. The first-order bone resorption process predicts that women who experience larger changes in BMD at the end of the treatment period will also have a faster recovery when elagolix treatment is stopped.

### Example 2: Simulation for Elagolix Dose Switching-Dose Escalation

A dose switching scenario was simulated where the elagolix dosing regimen beyond Month 3 was determined based on clinical response at Month 3 following start of treatment. Using dysmenorrhea (DYS) responder status, a median of 56% (95% confidence interval: 48-66%) required a dose increase from 150 mg QD to 200 mg BID at Month 3. Using non-menstrual pelvic pain (NMPP) responder status, a median of 49% (95% confidence interval: 41-60%) required a dose increase to 200 mg BID.

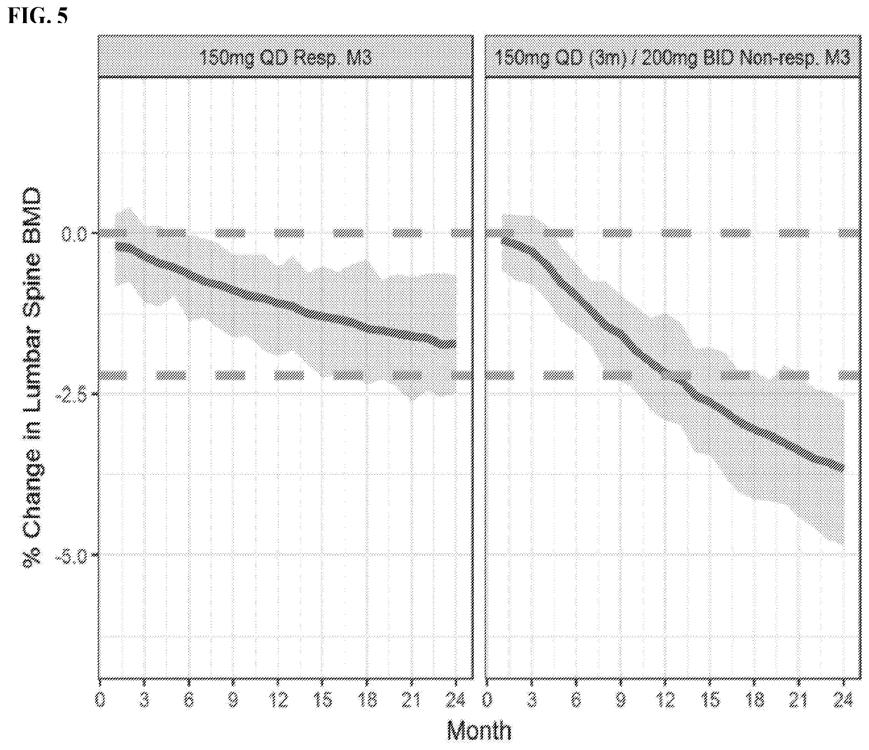

The predicted BMD changes and Z-scores in subjects who continued on 150 mg QD for 24 months or escalated to 200 mg BID at month 3 based on DYS responder status under a 70% dosing compliance assumption are shown in FIG. 5 and Table 3. Similar results were obtained when the simulations were performed using the NMPP responder status.

TABLE 3
Summary Statistics of Predicted Mean % BMD Changes and
Z-Scores for Elagolix Treatment Based on DYS Response.

| | | | | | | |
|---|---|---|---|---|---|---|
| | Mean °A | | Mean | | | |
| | Change | | Z-Score | | % subjects | % subjects |
| Month | in BMD | 95% CI | (ZS) | 95% CI | ZS < −1.5 | ZS < −2.0 |

150 mg QD, Responders at Month 3

| | | | | | | |
|---|---|---|---|---|---|---|
| 6 | −0.63 | −1.36-−0.0216 | 0.254 | −0.0261-−0.643 | 4.64 | 1.27 |
| 12 | −1.08 | −1.89-−0.519 | 0.211 | −0.0796-−0.588 | 5.31 | 1.57 |
| 24 | −1.71 | −2.49-−0.647 | 0.151 | −0.159-−0.521 | 6.25 | 2.11 |

150 mg QD (3 m) NR, Dose increase to 200 mg Month 3

| | | | | | | |
|---|---|---|---|---|---|---|
| 6 | −0.964 | −1.52-−0.467 | 0.225 | −0.0143-−0.471 | 5.00 | 1.53 |
| 12 | −2.16 | −2.91-−1.23 | 0.108 | −0.129-−0.359 | 6.37 | 2.00 |
| 24 | −3.66 | −4.85-−2.60 | −0.0383 | −0.266-−0.237 | 8.66 | 2.83 |

### Example 3: Simulation for Elagolix Dose Switching—Dose Reduction

A dose switching scenario was simulated where elagolix treatment started with 200 mg BID for 3 or 6 months followed by 150 mg QD for up to 24 months. Each simulated subject was treated with 200 mg BID during the first treatment period (3 or 6 months) and 150 mg QD during the second treatment period (21 or 18 months) for a total of 24 months and the % BMD change from baseline as well as Z-score were predicted over the entire treatment period.

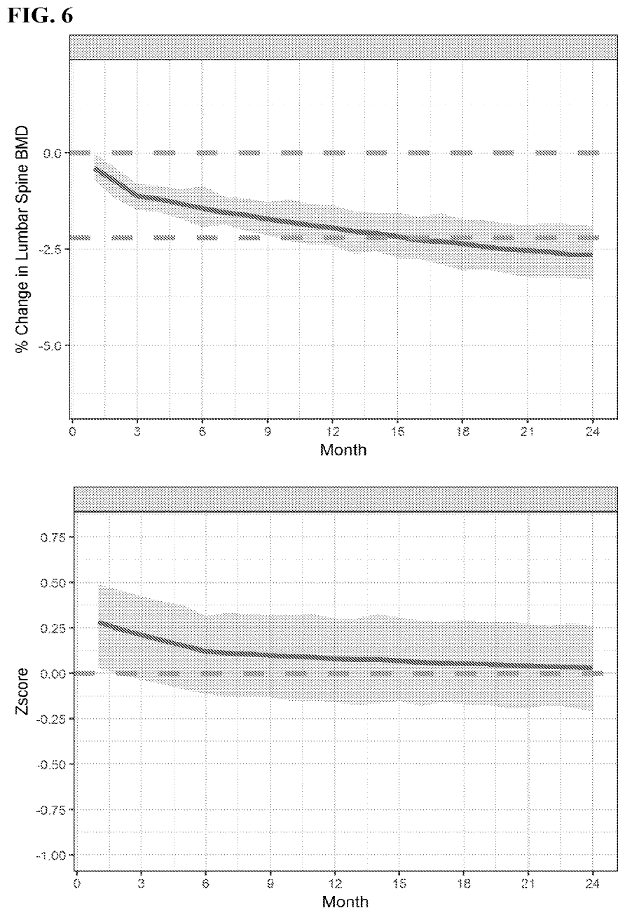

The predicted BMD changes and Z-scores in subjects who started with 200 mg BID for 3 months followed by 150 mg QD for up to 24 months under a 100% dosing compliance assumption are shown in FIG. 6 and Table 4.

TABLE 4
Summary Statistics of Predicted Mean % BMD Changes and
Z-Scores for Elagolix Dosing Starting with 200 mg BID
for 3 Months and Switching to 150 mg QD up to 24 Months.

| | Mean °A | | Mean | | | | |
|---|---|---|---|---|---|---|---|
| | Change | | Z-Score | | % subjects | % subjects | % subjects |
| Month | in BMD | 95% CI | (ZS) | 95% CI | ZS < −1.5 | ZS < −2.0 | ZS < −2.5 |
| 6 | −1.4 | −1.9-−0.85 | 0.122 | −0.11-0.32 | 6.0 | 1.9 | 0.43 |
| 12 | −1.9 | −2.4-−1.35 | 0.081 | −0.16-0.30 | 6.8 | 2.0 | 0.55 |
| 24 | −2.6 | −3.3-−1.89 | 0.032 | −0.21-0.26 | 8.1 | 2.8 | 0.65 |

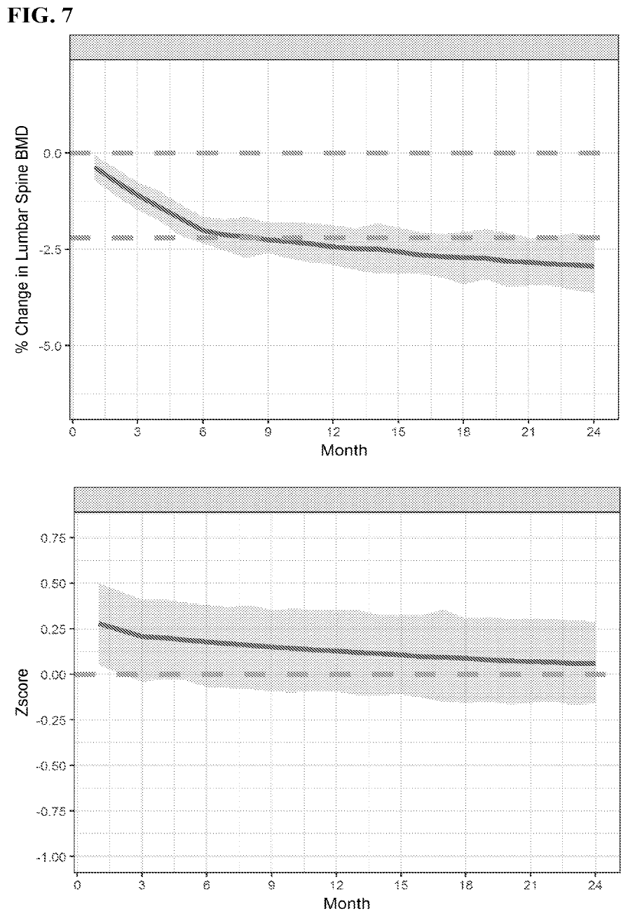

The predicted BMD changes and Z-scores in subjects who started with 200 mg BID for 3 or 6 months followed by 150 mg QD for up to 24 months under a 100% dosing compliance assumption are shown in FIG. 7 and Table 5.

TABLE 5
Summary Statistics of Predicted Mean % BMD Changes and
Z-Scores for Elagolix Dosing Starting with 200 mg BID
for 3 Months and Switching to 150 mg QD up to 24 Months.

| | Mean % | | Mean | | | | |
|---|---|---|---|---|---|---|---|
| | Change | | Z-Score | | % subjects | % subjects | % subjects |
| Month | in BMD | 95% CI | (ZS) | 95% CI | ZS < −1.5 | ZS < −2.0 | ZS < −2.5 |
| 6 | −2.0 | −2.3-−1.7 | 0.18 | −0.069-0.38 | 5.7 | 1.6 | 0.33 |
| 12 | −2.4 | −2.9-−1.9 | 0.13 | −0.095-0.36 | 6.2 | 1.9 | 0.50 |
| 24 | −2.9 | −3.6-−2.2 | 0.06 | −0.156-0.29 | 7.8 | 2.6 | 0.57 |

### Example 4: Twelve Month Study with 300 mg BID

This study was a randomized, double-blind, multicenter, extension study designed to evaluate the efficacy and safety of elagolix alone and in combination with an exemplary low-dose hormone (add-back) therapy (estradiol (“E2”) 1.0 mg/norethindrone acetate (“NETA”) 0.5 mg) in premenopausal women with heavy menstrual bleeding associated with uterine fibroids for up to 12 months total (an initial 6 months on active treatment in one of two replicate initial (i.e., pivotal) studies and an additional 6 months in this extension study.

Subjects who received elagolix 300 mg twice daily or elagolix 300 mg twice daily in combination with E2/NETA in the initial studies continued to receive the same treatment (n=94 and 206, respectively) while subjects who received placebo in the initial studies were randomized in an equal ratio to one of the two treatment groups (elagolix 300 mg BID (n=56) or elagolix 300 mg BID+E2/NETA (n=54)).

The primary efficacy endpoint was the percentage of subjects meeting the responder criteria: a) menstrual blood loss (MBL) volume <80 mL during the final, AND b) 50% or greater reduction in MBL volume from baseline to the final month. A subject who prematurely discontinued the study drug due to adverse events, “lack of efficacy,” or “requires surgery or invasive intervention for treatment of uterine fibroids” was considered a non-responder regardless of whether she met the response criteria. For this uncontrolled extension study, with the exception of the 12 month BMD, there are no statistical tests of comparison of efficacy or safety endpoints performed (i.e., no p-values to report). The safety analysis was conducted on the full patient set.

An overview of the results from the initial study and extension study is below.

Primary Endpoint Results at Final Month Across the Studies

Responder Rate

| | Elagolix 300 mg BID + | |
|---|---|---|
| Study | Add-Back (E2/NETA) | PBO |
| Initial Study I | 68.5% (p < 0.001) | 8.7% |
| | n = 206 | n = 102 |
| Initial Study II | 76.2% (p < 0.001) | 10.1% |
| | n = 189 | n = 94 |
| Extension Study | 87.9% | N/A |
| | n = 206 | |

Elagolix (300 mg twice daily), in combination with low-dose hormone (add-back) therapy (estradiol 1.0 mg/norethindrone acetate 0.5 mg), reduced heavy menstrual bleeding with 87.9 percent of women with uterine fibroids achieving clinical response.

Hypoestrogenic effects, such as hot flush and reduction in bone mineral density, from elagolix treatment were observed in the studies. Evaluation of bone mineral density (BMD) showed use of add-back therapy may reduce BMD decreases when used with elagolix.

The results from this study demonstrate the continued efficacy of elagolix for up to twelve months in the management of heavy menstrual bleeding associated with uterine fibroids with no new safety signals.

It is understood that the foregoing detailed description and accompanying examples are merely illustrative and are not to be taken as limitations upon the scope of the invention, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, formulations, or methods, or any combination of such changes and modifications of use of the invention, may be made without departing from the spirit and scope thereof

All references (patent and non-patent) cited above are incorporated by reference into this patent application. The discussion of those references is intended merely to summarize the assertions made by their authors. No admission is made that any reference (or a portion of any reference) is relevant prior art (or prior art at all). Applicant reserves the right to challenge the accuracy and pertinence of the cited references.

What is claimed is:

1. A method for treating a female subject with 4-((R)-2-[5-(2-fluoro-3-methoxy-phenyl)-3-(2-fluoro-6-trifluoromethyl-benzyl)-4-methyl-2,6-di oxo-3,6-dihydro-2H-pyrimi din-1-yl1-1-phenyl-ethylamino)-butyric acid (Compound A) or a pharmaceutically acceptable salt thereof, the method comprising:
administering Compound A or a pharmaceutically acceptable salt thereof to the subject according to a first dosing schedule during a first treatment period; and
administering Compound A or a pharmaceutically acceptable salt thereof to the subject according to a second dosing schedule during a second treatment period, wherein the second dosing schedule comprises a lower dose and/or less frequent administration than the first dosing schedule.

2. The method of claim 1, wherein the first treatment period is about three months or about six months.

3. The method of claim 1, wherein the second treatment period is from about eighteen to about twenty four months.

4. The method of claim 1, wherein the subject is suffering from endometriosis, adenomyosis, polycystic ovary syndrome (PCOS), or uterine fibroids.

5. The method of claim 1, wherein the first dosing schedule comprises twice daily administration of Compound A or a pharmaceutically acceptable salt thereof.

6. The method of claim 1, wherein the second dosing schedule comprises once daily administration of Compound A or a pharmaceutically acceptable salt thereof.

7. The method of claim 1, wherein Compound A or a pharmaceutically acceptable salt thereof is sodium 4-((R)-2-[5-(2-fluoro-3-methoxy-phenyl)-3-(2-fluoro-6-trifluoromethyl benzyl 1)-4-methyl-2,6-dioxo-3,6-dihydro-2H-pyrimidin-1-yl1-1-phenyl-ethylamino) butanoate.

8. A method for treating a female subject with 4-((R)-2-[5-(2-fluoro-3-methoxy-phenyl)-3-(2-fluoro-6-trifluoromethyl-benzyl)-4-methyl-2,6-dioxo-3,6-dihydro-2H-pyrimidin-1yl]-1-phenyl-ethylamino)-butyric acid (Compound A) or a pharmaceutically acceptable salt thereof, the method comprising:
administering Compound A or a pharmaceutically acceptable salt thereof to the subject according to a first dosing schedule during a first treatment period, wherein the first dosing schedule comprises twice daily administration of about 200 mg of Compound A or a pharmaceutically acceptable salt thereof; and
subsequently administering Compound A or a pharmaceutically acceptable salt thereof to the subject according to a second dosing schedule during a second treatment period, wherein the second dosing schedule comprises once daily administration of about 150 mg of Compound A or a pharmaceutically acceptable salt thereof.

9. The method of claim 8, wherein the first treatment period is not more than about six months.

10. The method of claim 8, wherein the second treatment period is from about eighteen to about twenty four months.

11. The method of claim 8, wherein the subject is suffering from endometriosis, adenomyosis, polycystic ovary syndrome (PCOS), or uterine fibroids.

12. The method of claim 8, wherein Compound A or a pharmaceutically acceptable salt thereof is sodium 4-((R)-2-[5-(2-fluoro-3-methoxy-phenyl)-3-(2-fluoro-6-trifluoromethyl-benzyl)-4-methyl-2,6-di oxo-3,6-dihydro-2H-pyrimidin-1-yl]-1-phenyl ethylamino) butanoate.

13. A method for reducing the rate of bone mineral density loss in a subject treated with 4-((R)-2-[5-(2-fluoro-3-methoxy-phenyl)-3-(2-fluoro-6-trifluoromethyl-benzyl)-4-methyl-2,6-dioxo-3,6-dihydro-2H-pyrimidin-1-yl]-1-phenyl-ethylamino)-butyric acid (Compound A) or a pharmaceutically acceptable salt thereof, the method comprising
administering Compound A or a pharmaceutically acceptable salt thereof to the subject according to a first dosing schedule during a first treatment period, wherein the first dosing schedule is associated with a first rate of bone mineral density loss;
subsequently administering Compound A or a pharmaceutically acceptable salt thereof to the subject according to a second dosing schedule during a second treatment period, wherein the second dosing schedule is associated with a second rate of bone mineral density loss that is reduced relative to the first rate of bone mineral density loss.

14. The method of claim 13, wherein the second dosing schedule comprises a reduced dosage amount, dosing frequency, and/or total daily dose relative to the first treatment period.

15. The method of claim 13, wherein the first treatment period is about three months or about six months.

16. The method of claim 13, wherein the second treatment period is from about eighteen to about twenty four months.

17. The method of claim 13, wherein the subject is suffering from endometriosis, adenomyosis, polycystic ovary syndrome (PCOS), or uterine fibroids.

18. The method of claim 13, wherein Compound A or a pharmaceutically acceptable salt thereof is sodium 4-((R)-2-[5-(2-fluoro-3-methoxy-phenyl)-3-(2-fluoro-6-trifluoromethyl-benzyl)-4-methyl-2,6-dioxo-3,6-dihydro-2H-pyrimidin-1-yl]-1-phenyl-ethylamino) butanoate.

19. A method for treating uterine fibroids in a female subject in need thereof, the method comprising:
administering 4-((R)-2-[5-(2-fluoro-3-methoxy-phenyl)-3-(2-fluoro-6-trifluoromethyl-benzyl)-4-methyl-2,6-dioxo-3,6-dihydro-2H-pyrimidin-1-yl]-1-phenyl-ethylamino)-butyric acid (Compound A) or a pharmaceutically acceptable salt thereof to the subject during a treatment period, wherein the treatment period lasts for more than six months; and
co-administering a hormone add-back therapy during the treatment period.

20. The method of claim 19, wherein the hormone add-back therapy comprises estradiol and norethindrone acetate.
